(19) United States
(12) Patent Application Publication
(10) Pub. No.: US 2026/0330850 A1
(43) Pub. Date: Oct. 22, 2026

(54) METHODS, SYSTEMS, AND PROGRAM PRODUCTS FOR CLASSIFYING DIFFERENT SPECIES OF MICROBES

(71) Applicant: The Research Foundation for the State University of New York, Albany, NY (US)

(72) Inventor: Alex Valm, Albany, NY (US)

(21) Appl. No.: 19/651,375

(22) Filed: Apr. 17, 2026

Related U.S. Application Data
(60) Provisional application No. 63/790,511, filed on Apr. 17, 2025.

(51) Int. Cl. G06V 20/69; G06V 10/764; G06V 10/774; G16B 40/20

(52) U.S. Cl. CPC G06V 20/698; G06V 10/764; G06V 10/774; G16B 40/20

(57) ABSTRACT

Methods for classifying different microbes are disclosed. The method includes receiving an image of a mixture of a plurality of fluorophore deoxyribonucleic acid (DNA) probes and an organic sample including a plurality of species of microbes, wherein each of the plurality of fluorophore-DNA probes corresponds to a predetermined species of microbe. The method also includes analyzing the received image of the mixture to identify a bond between at least one fluorophore-DNA probe of the plurality of fluorophore-DNA probes and at least one species of microbe of the plurality of species of microbes. The method further includes classifying the at least one species of microbe based upon the predetermined species of microbe associated with the at least one fluorophore-DNA probe bonded with the at least one species of microbe.

### CROSS-REFERENCE TO RELATED APPLICATION

This application claims the benefit of U.S. Provisional Patent Application No. 63/790,511, filed Apr. 17, 2025, the entirety of which is hereby incorporated herein by this reference.

### GOVERNMENT RIGHTS

This invention was made with government support under Grant No. DE030927 awarded by the National Institutes of Health. The government has certain rights in the invention.

### BACKGROUND

The disclosure relates generally to classifying different microbes, and more particularly, to differentiating and classifying various microbes in a single sample using deoxyribonucleic acid (DNA) probes, synthesized fluorophore-based dyes, and machine learning.

The spatial organization of microbial communities is a critical determinant of host-microbe interactions. However, identifying specific species within these communities is challenging due to similarities in their genetic sequences. Microbes with highly similar DNA sequences, such as those in the 16S rRNA gene, can be difficult to distinguish from one another. This similarity in DNA sequences can lead to cross-hybridization, where a probe or primer intended to bind to a specific species binds to a non-target species instead. As a result, traditional methods of microbial classification, such as fluorescence in situ hybridization (FISH), can be limited in their ability to accurately identify and distinguish between closely related species.

The limitations of traditional classification methods can have significant implications for our understanding of microbial communities and their role in various ecosystems. In communities with phylogenetically closely related organisms, the design of species-specific FISH probes for discriminating community members is challenging due to the high level of homology in the 16S rRNA genes. This can lead to inaccurate or incomplete classification of microbes, which can in turn impact our ability to understand the complex interactions within microbial communities.

Therefore, there is a need for new methods and approaches that can accurately and reliably classify microbes, even those with highly similar DNA sequences. By developing such methods, researchers can gain a deeper understanding of microbial communities and their role in shaping ecosystem processes.

### BRIEF DESCRIPTION

In some aspects, the techniques described herein relate to a method, including: receiving an image of a mixture of a plurality of fluorophore deoxyribonucleic acid (DNA) probes and an organic sample including a plurality of species of microbes, wherein each of the plurality of fluorophore-DNA probes corresponds to a predetermined species of microbe; analyzing, by one or more processors, the received image of the mixture to identify a bond between at least one fluorophore-DNA probe of the plurality of fluorophore-DNA probes and at least one species of microbe of the plurality of species of microbes; and classifying, by the one or more processors, the at least one species of microbe based upon the predetermined species of microbe associated with the at least one fluorophore-DNA probe bonded with the at least one species of microbe.

In other aspects, the techniques described herein relate to a system including: one or more processors coupled to non-transitory memory, the one or more processors configured to: receive an image of a mixture of a plurality of fluorophore deoxyribonucleic acid (DNA) probes and an organic sample including a plurality of species of microbes, wherein each of the plurality of fluorophore-DNA probes corresponds to a predetermined species of microbe; analyze the received image of the mixture to identify a bond between at least one fluorophore-DNA probe of the plurality of fluorophore-DNA probes and at least one species of microbe of the plurality of species of microbes; and classify the at least one species of microbe based upon the predetermined species of microbe associated with the at least one fluorophore-DNA probe bonded with the at least one species of microbe.

In additional aspects, the techniques described herein relate to a non-transitory computer-readable medium including one or more instructions stored thereon and executable by a processor to: receive an image of a mixture of a plurality of fluorophore deoxyribonucleic acid (DNA) probes and an organic sample including a plurality of species of microbes, wherein each of the plurality of fluorophore-DNA probes corresponds to a predetermined species of microbe; analyze the received image of the mixture to identify a bond between at least one fluorophore-DNA probe of the plurality of fluorophore-DNA probes and at least one species of microbe of the plurality of species of microbes; and classify the at least one species of microbe based upon the predetermined species of microbe associated with the at least one fluorophore-DNA probe bonded with the at least one species of microbe.

The illustrative aspects of the present disclosure are designed to solve the problems herein described and/or other problems not discussed.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 shows a schematic diagram of a system for classifying various microbes from captured images, according to embodiments of the disclosure.

FIG. 2A shows a flow chart illustrating processes for classifying various microbes, according to embodiments of the disclosure.

FIG. 2B shows a flow chart illustrating processes for training a machine learning model used to classify various microbes, according to embodiments of the disclosure.

FIG. 3 shows a table including information relating to training microbes and training fluorophore-DNA probes used to train a machine learning model, according to embodiments of the disclosure.

FIG. 4 shows a plurality of images captured of various mixtures of the training microbes and the training fluorophore-DNA probes included in the table of FIG. 3, according to embodiments of the disclosure.

FIG. 5 shows spectra profiles for various mixtures of the training microbes and the training fluorophore-DNA probes using a range of wavelengths for excitation of the fluorophore-based dyes, according to embodiments of the disclosure.

FIG. 6 shows a confusion matrix depicting the accuracy of classifying the training microbes using the training fluorophore-DNA probes, according to embodiments of the disclosure.

FIG. 7 shows various adjusted images depicting each training microbe and the corresponding training fluorophore-DNA probes used to classify the training microbes, according to embodiments of the disclosure.

FIG. 8 shows various raw and fluorophore-based dye highlighted images of various microbes, according to embodiments of the disclosure.

FIG. 9 shows a fluorophore-based dye highlighted image of a mixture of the various microbes included in FIG. 8, according to embodiments of the disclosure.

FIG. 10 shows a table identifying various microbes, the accuracy in detecting the microbes using conventional linear unmixing, and the machine learning-based classification processes of FIGS. 2A and 2B, according to embodiments of the disclosure.

FIG. 11 shows a schematic of a system for training artificial intelligence models using training images of a mixture of training fluorophore-DNA probes and training microbes, according to embodiments of the disclosure.

It is noted that the drawings of the disclosure are not to scale. The drawings are intended to depict only typical aspects of the disclosure, and therefore should not be considered as limiting the scope of the disclosure. In the drawings, like numbering represents like elements between the drawings.

Additionally, the patent or application file contains at least one drawing executed in color. Copies of this patent or patent application publication with color drawing(s) will be provided by the Office upon request and payment of the necessary fee.

### DETAILED DESCRIPTION

As an initial matter, in order to clearly describe the current disclosure, it will become necessary to select certain terminology when referring to and describing relevant components within the disclosure. When doing this, if possible, common industry terminology will be used and employed in a manner consistent with its accepted meaning. Unless otherwise stated, such terminology should be given a broad interpretation consistent with the context of the present application and the scope of the appended claims. Those of ordinary skill in the art will appreciate that often a particular component may be referred to using several different or overlapping terms. What may be described herein as being a single part may include and be referenced in another context as consisting of multiple components. Alternatively, what may be described herein as including multiple components may be referred to elsewhere as a single part.

As discussed herein, the disclosure relates generally to classifying different microbes, and more particularly, to differentiating and classifying various microbes in a single sample using deoxyribonucleic acid (DNA) probes, synthesized fluorophore-based dyes, and machine learning.

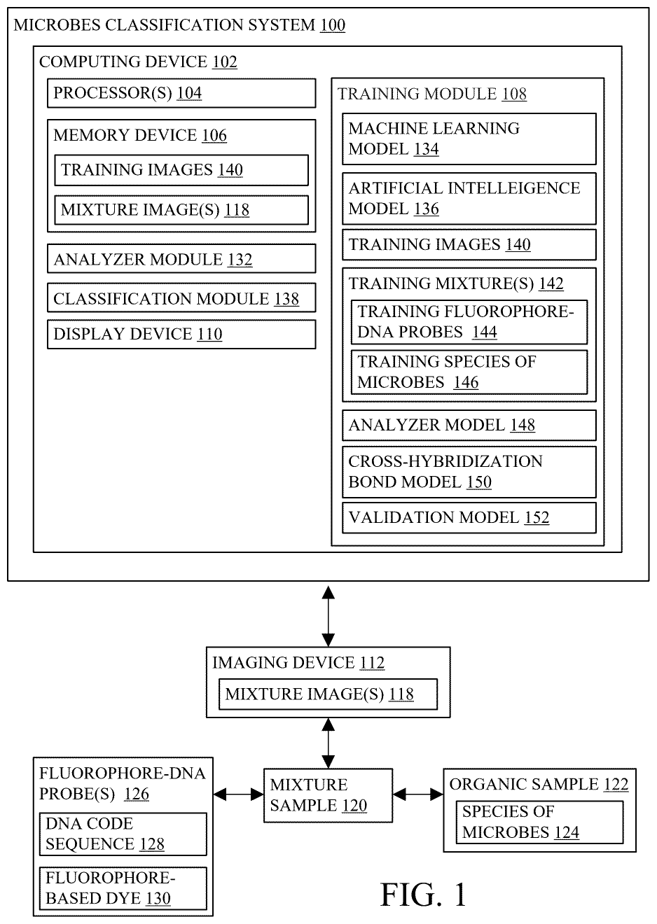

These and other embodiments are discussed below with reference to FIGS. 1-11. However, those skilled in the art will readily appreciate that the detailed description given herein with respect to these Figures is for explanatory purposes only and should not be construed as limiting.

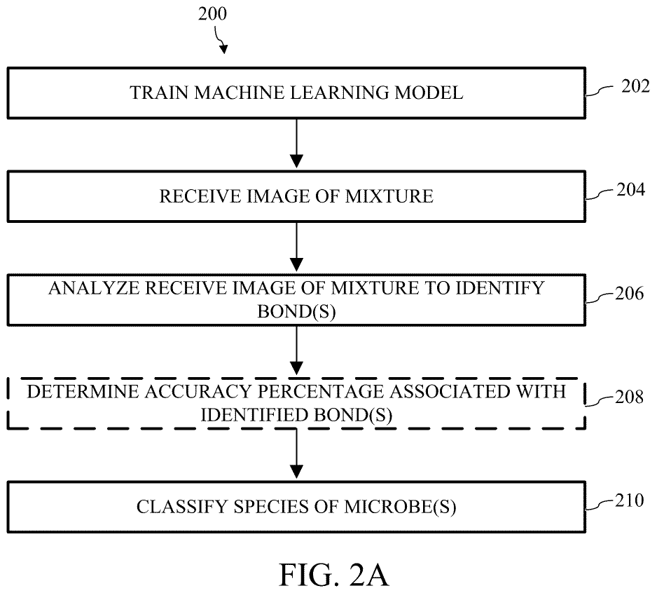

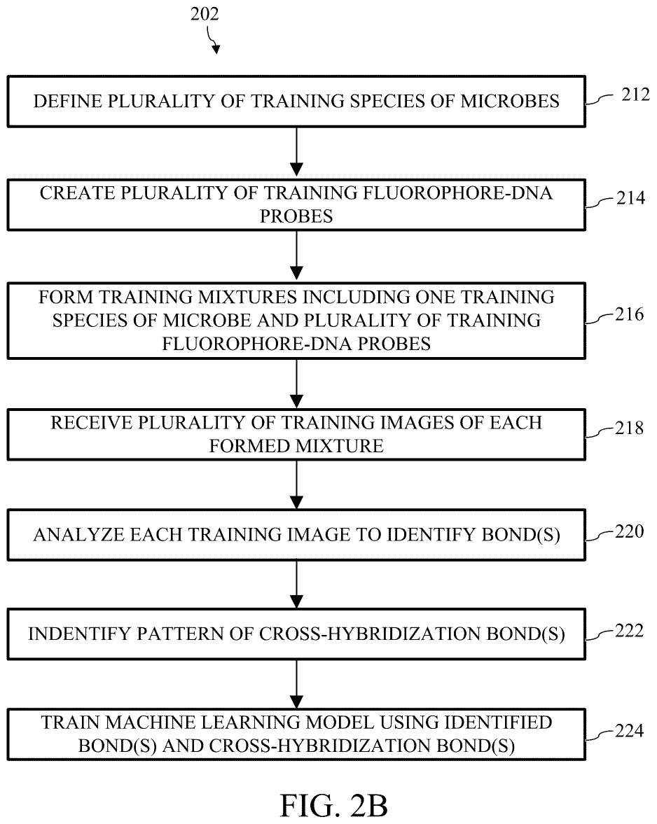

FIG. 1 is a schematic diagram of a microbes classification system 100 (hereafter, system “100”). The system 100 shown in FIG. 1 is configured to perform the processes 200 and 202, shown and discussed herein with respect to FIGS. 2A and 2B, for classifying various microbes in a single sample using deoxyribonucleic acid (DNA) probes, synthesized fluorophore-based dyes, and machine learning.

With reference to FIG. 1, system 100 includes a computing device 102 including various components, modules, and/or models included therein and/or in communication therewith. For example, computing device 102 of system 100 includes at least one processor 104, a memory device 106, a training module 108, a display device 110, and various distinct modules, models, and/or devices, as discussed herein. The various systems, components, and/or modules serve as inputs to and receive outputs from various other components of system 100 and/or imaging device 112, as discussed herein. In other examples, system 100 may include more, fewer, or different components or systems, and each of the components or system(s) may include more, fewer, or different components or models. Additionally, the systems and components shown may be combined or divided in various ways.

In exemplary embodiments, the processor 104 of system 100 may be embodied as one or more of a data processor, a microcontroller, a microprocessor, a digital signal processor, a logic circuit, a programmable logic array, or one or more other devices for controlling system 100 in response to one or more of the system inputs. System 100 may include a single microprocessor or multiple microprocessors that may include means for classifying microbes and/or training models used for classification, as discussed herein. Numerous commercially available microprocessors can be configured to perform the functions of system 100. It should be appreciated that system 100 could include a general machine controller capable of controlling numerous other machine functions. Additionally, a special-purpose machine controller can be provided in some of the examples described herein. Further, system 100, or portions thereof, may be located remote from system 100 and/or computing device 102. For example, one or more features of training module 108 could be located remote from computing device 102. Various other known circuits may be associated with system 100, including signal-conditioning circuitry, communication circuitry, actuation circuitry, and other appropriate circuitry.

Memory device 106 of system 100 may store data and/or software routines that may assist system 100 in performing its functions, such as the functions of classifying different species of microbes (e.g., process 200 described herein with respect to FIG. 2A), and/or training artificial intelligence models/machine learning models of training module 108 (e.g., process 202 described herein with respect to FIG. 2B). Further, the memory device 106 may also store data received from various inputs associated with system 100, such as training images, as well as mixture images. For example, and as discussed herein, memory device 106 may store training images to be used in training artificial intelligence models/machine learning models of training module 108, as well as mixture images currently undergoing analysis to classify species of microbes included in an imaged sample.

Imaging device 112 is in communication with and/or communicatively coupled to system 100, and is configured to capture at least one mixture image 118 of a mixture sample 120 including at least one species of microbes that require classification by system 100. In exemplary embodiments, imaging device 112 is formed as any suitable device configured to generate, render, and/or capture a maximum intensity projection (MIP) image for analysis by system 100, including, but not limited to, electron microscopy instruments, ultrasound imaging devices, computer tomography (CT) scanners, position emission tomography (PET) devices, and the like. Additionally, although discussed herein as rendering and/or capturing an MIP image of mixture sample 120, it is understood that imaging device 112 can generate any suitable mixture image 118 to be used by system 100 for classifying microbes in mixture sample 120 that is captured in mixture image 118. In a non-limiting example, and as discussed herein, mixture image 118 provided to system 100 includes a single image of mixture sample 120, which is analyzed by system 100 to classify the various species of microbes included within the mixture sample 120.

Mixture sample 120 is formed from a plurality of distinct components, materials, and/or products that undergo processes discussed herein (e.g., process 200) for classifying distinct species of microbes included in mixture sample 120. More specifically, mixture sample 120 is formed, in part, by an organic sample 122 including at least one species of microbes 124. In exemplary embodiments, organic sample 122 can include a biological fluid or tissue extracted from an organism, for example, a gut microbiota, that includes a plurality of species of microbes 124. Additionally, the plurality of species of microbes 124 can include, but are not limited to, bacteria, viruses, fungi, protozoa, and the like. Continuing the example where organic sample 122 is formed as a gut microbiota, the plurality of species of microbes 124 can include various bacteria found within the gut microbiota (e.g., Aeromonas caviae (UGB3607), Stenotrophomonas maltophilia (UGB3616), etc.). As it is understood, prior to imaging using imaging device 112 and processing by system 100, the various species of microbes 124 included in organic sample 122 are not readily detectable, differentiable, and/or classifiable by mere imaging and/or observation.

Additionally, in the non-limiting example, mixture sample 120 is also formed from a plurality of fluorophore deoxyribonucleic acid (DNA) probes 126 (hereafter, “fluorophore-DNA probes 126”). That is, a plurality of fluorophore-DNA probes 126 and organic sample 122, including species of microbes 124, are mixed together to form mixture sample 120 prior to imaging device 112 capturing mixture image 118 for processing by system 100. Each of the plurality of fluorophore-DNA probes 126 included in mixture sample 120 corresponds to a predetermined species of microbe. The predetermined species of microbe corresponding with each fluorophore-DNA probe 126 may include at least one of the various species of microbes 124 included in organic sample 122 of mixture sample 120. In exemplary embodiments, each fluorophore-DNA probe 126 includes a unique DNA code sequence 128 that corresponds to, is associated with, and/or is complementary to a DNA code sequence (e.g., 16S rRNA gene sequence) of a predetermined species of microbe. In the embodiment, the DNA code sequence 128 for fluorophore-DNA probe 126 is created, determined, and/or produced to correspond with the DNA code sequence of a single, predetermined species of microbe, and more specifically, is formed and/or configured to bond with the DNA code sequence of the single, predetermined species of microbe when mixed together (e.g., mixture sample 120).

Furthermore, each of the plurality of fluorophore-DNA probes 126 included in mixture sample 120 also includes a uniquely colored, fluorophore-based dye 130. More specifically, each fluorophore-DNA probe 126 includes a fluorophore-based dye 130 having a color that is unique and/or specific to the individual fluorophore-DNA probe 126. As such, no fluorophore-DNA probes 126, including distinct and/or unique DNA code sequence 128, include the same color of fluorophore-based dye 130 included therein. Fluorophore-based dyes 130 are formed from any suitable fluorescent chemical compounds to be used as markers and/or are configured to absorb light at specific wavelengths and re-emit at distinct wavelengths for detection during imaging and/or inspection (e.g., microscopy). As discussed herein, the use of uniquely colored, fluorophore-based dye 130 in each of the plurality of fluorophore-DNA probe 126 aids in the classification and/or distinguishing of the plurality of species of microbes 124 included in mixture sample 120. Furthermore, it is to be understood that the unique DNA code sequence 128 and the uniquely colored, fluorophore-based dye 130 forming each of the plurality of fluorophore-DNA probes 126 are chemically conjugated to one another in forming fluorophore-DNA probes 126.

In creating mixture sample 120, a plurality of fluorophore-DNA probes 126 are mixed with organic sample 122. The plurality of fluorophore-DNA probes 126 mixed with organic sample 122 to form mixture sample 120 may be preselected and/or predetermined based on characteristics and/or properties relating to organic sample 122 and/or species of microbes 124. For example, wherein organic sample 122 includes a gut microbiota, the plurality of fluorophore-DNA probes 126 may be a predetermined collection of probes that are likely to correspond to, bond with, and/or complement microbes that are typically included in the gut microbiota. As such, the plurality of fluorophore-DNA probes 126 included in mixture sample 120 are customizable to improve the bonding with species of microbes 124 included in organic sample 122, and in turn, improve the classification of the species of microbes 124 by system 100. In other exemplary embodiments, the plurality of fluorophore-DNA probes 126 included in mixture sample 120 include a predetermined and/or pre-established group of probes that are independent from organic sample 122. In this exemplary embodiment, multiple groups of fluorophore-DNA probes 126 can be mixed with multiple samples of the same organic sample 122 and processed by system 100 to ensure all species of microbes 124 included in organic sample 122 are classified, as discussed herein.

As discussed herein, mixture images of mixture sample 120 are captured and/or generated by imaging device 112 and provided to and/or received by system 100 to classify at least one of the species of microbes 124 included in mixture sample 120. Distinct modules, models, and/or components included within system 100 perform distinct processes to aid in classifying species of microbes 124. Additionally, at least a portion of the distinct modules, models, and/or components included within system 100 communicate with one another in order to facilitate the classification of species of microbes 124 using mixture images 118.

In exemplary embodiments, analyzer module 132 interacts and/or communicates with various components and/or modules to analyze mixture image(s) 118 and identify bonds between fluorophore-DNA probes 126 and one species of microbe of the plurality of species of microbes 124 included in organic sample 122. For example, analyzer module 132 communicates with imaging device 112 to receive mixture image(s) 118. Additionally, analyzer module 132 is in communication with memory device 106, and is configured to provide and/or store mixture image(s) 118 within memory device 106. Analyzer module 132 is also configured to interact with various models within training module 108, including machine learning model 134 and artificial intelligence model 136, respectively. Analyzer module 132 of system 100, with the aid of training module 108, is configured to analyze the received mixture image(s) 118 to identify any bonds between fluorophore-DNA probes 126 and one species of microbe of the plurality of species of microbes 124 included in organic sample 122. More specifically, analyzer module 132 is configured to identify a bond, if any, between at least one fluorophore-DNA probe of the plurality of fluorophore-DNA probes 126 and at least one species of microbe of the plurality of species of microbes 124 mixed together to form mixture sample 120. As discussed herein with respect to process 200 shown in FIG. 2A, and by way of example, analyzer module 132 is configured to identify a first bond between a first fluorophore-DNA probe of the plurality of fluorophore-DNA probes 126 and a first species of microbes of the plurality of species of microbes 124, and/or identify a second bond between the first or same fluorophore-DNA probe of the plurality of fluorophore-DNA probes 126 and a second species of microbes of the plurality of species of microbes 124, where the second, bonded species is distinct from the first bonded species. Additionally, and as discussed herein with respect to process 200, analyzer module 132 of system 100, with the aid of training module 108, is configured to determine an accuracy percentage associated with the first bond and the second bond. The accuracy percentage aids in classifying the bonded species of microbe included in the plurality of species of microbes 124.

Classification module 138 similarly interacts and/or communicates with various components and/or modules to classify at least one species of microbes 124 included in organic sample 122 and bonded to at least one fluorophore-DNA probe 126 within mixture sample 120. For example, classification module 138 communicates with analyzer module 132 to process information and/or data relating to the identified bonds (if any) formed between fluorophore-DNA probes 126 and species of microbes 124 included in organic sample 122. Specifically, and based on known DNA code sequences 128 and/or uniquely colored, fluorophore-based dye 130, specific fluorophore-DNA probes 126 are identified by analyzer module 132 based on identified bonding, and such data or information is provided from analyzer module 132 to classification module 138 for additional processing (e.g., process 200). Additionally, classification module 138 is also configured to interact with various models within training module 108, including machine learning model 134 and artificial intelligence model 136, respectively. Classification module 138 of system 100, with the aid of training module 108, is configured to receive data, information, and/or input from analyzer module 132 and classify the at least one bonded species of microbe of the plurality of species of microbes 124 included in organic sample 122. More specifically, classification module 138 is configured to classify, distinguish, and/or identify the bonded species of microbe based upon the predetermined species of microbe associated with the fluorophore-DNA probe 126 bonded with the species of microbe. For example, when it is determined or identified by analyzing mixture image 118 that only a single fluorophore-DNA probe 126 is bonded with a single species of microbe included in the plurality of species of microbes 124, classification module 138 of system 100 can classify the bonded, single species of microbe as the predetermined and/or predefined microbe that corresponds and/or is associated with the single, bonded fluorophore-DNA probe 126. However, and continuing the example above, where a first bond and a second bond exist between a single fluorophore-DNA probe 126 and two distinct species of microbes, classification module 138 of system 100, with the aid of training module 108, can classify at least one of the two distinct, bonded species of microbes. In the exemplary embodiment, classification module 138 can further analyze data or information generated by analyzer module 132, including but not limited to the accuracy percentage associated with each bond, and classify at least one of the bonded species of microbes based on the accuracy percentage associated with each bond. Where the accuracy percentage for the first bond between the single fluorophore-DNA probe 126 and the first species of microbe is greater than the accuracy percentage for the second bond between the single fluorophore-DNA probe 126 and the second, distinct species of microbe, classification module 138 of system 100 can classify the bonded, first species of microbe as the predetermined and/or predefined microbe that corresponds and/or is associated with the single, bonded fluorophore-DNA probe 126.

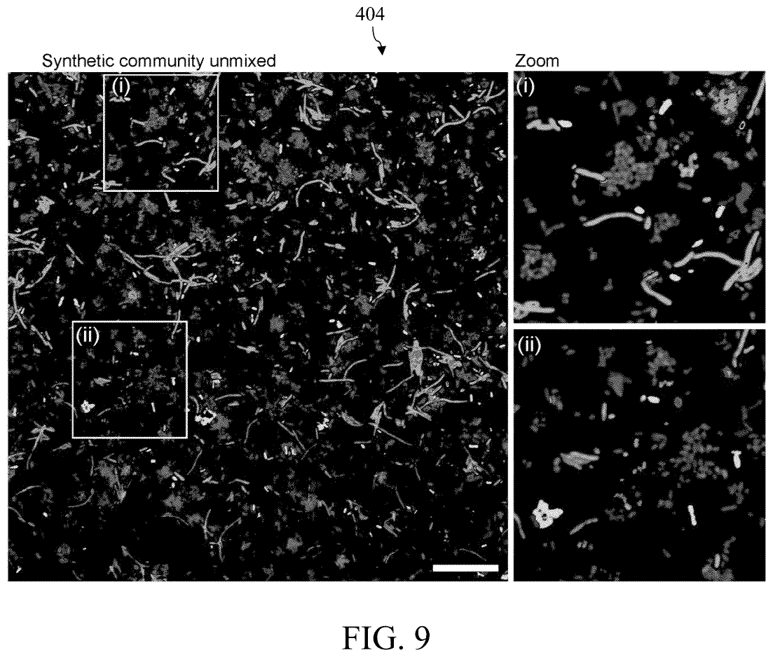

Display device 110 of system 100 includes any suitable device that is in communication with classification module 138, memory device 106, and/or imaging device 112. Display device 110 of system 100 is configured to display data and/or information determined relating to mixture sample 120, as determined or detected by imaging device 112, analyzer module 132, and/or classification module 138, as discussed herein. For example, display device 110, formed as a computer monitor or screen, is configured to display mixture image(s) 118 captured by imaging device 112, as well as provide visual feedback (e.g., list) of all identified and/or classified species of microbes 124 included in mixture sample 120, as determined using training module 108, analyzer module 132, and/or classification module 138, respectively. Additionally, and as discussed herein (see e.g., FIG. 9), a modified, adjusted, and/or altered mixture image(s) 118 is visually provided on display device 110 depicting each classified species of microbes 124 having a distinct color as defined by the uniquely colored, fluorophore-based dye 130 included in fluorophore-DNA probe 126 corresponding to, associated with, and/or bonded to the classified species of microbes 124.

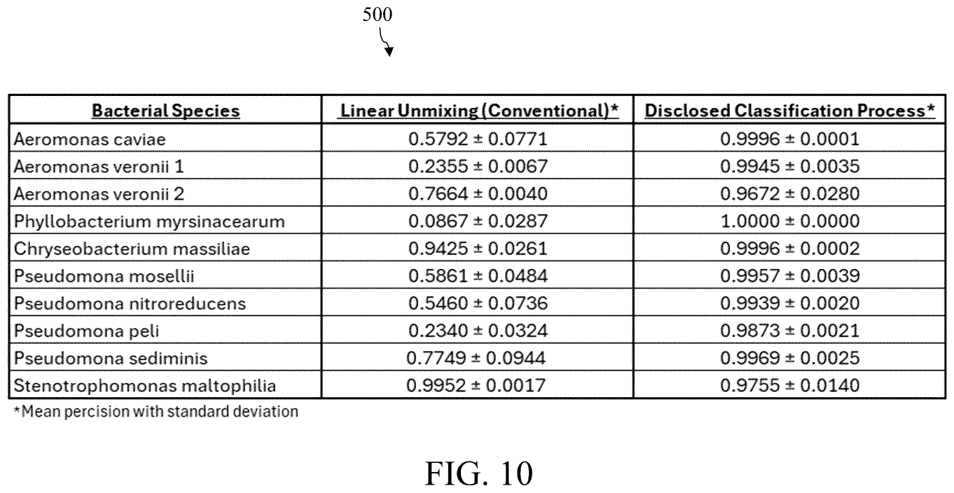

Training module 108 of system 100 may interact and/or communicate with various components and/or modules included within system 100 to aid analyzer module 132 and classification module 138 in classifying species of microbes 124 included in mixture sample 120. For example, training module 108 may communicate with memory device 106, imaging device 112, analyzer module 132 and/or classification module 138 to obtain data to aid in the classification process discussed herein. Additionally, training module 108 includes various components that communicate with one another to perform processes for training machine learning model 134 to aid analyzer module 132 and/or classification module 138 in classifying species of microbes, as discussed herein. In the exemplary embodiment, training module 108 includes machine learning model 134, artificial intelligence model 136, training images 140 (also stored in memory device 106), analyzer model 148, cross-hybridization bond model 150, and/or validation model 152. In addition to training module 108, and more specifically machine learning model 134, being configured to aid system 100 in the classification of species of microbes 124, training module 108 and the various components or models included therein, are configured to train machine learning model 134/artificial intelligence model 136 by performing process 202 as described herein with respect to FIG. 2B. Training machine learning model 134 improves system's 100 ability to accurately classify species of microbes 124 using fluorophore-DNA probes 126 over conventional classification processes (see, FIG. 10).

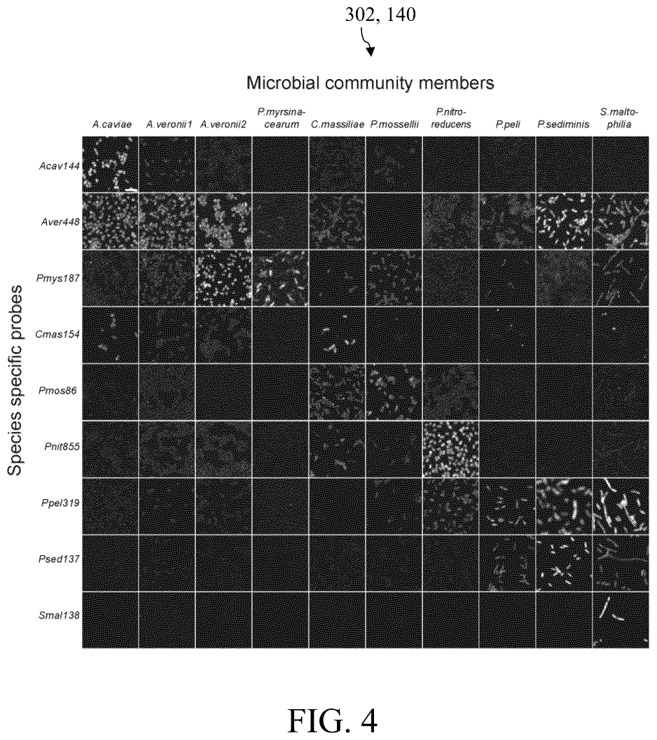

An initial training of machine learning model 134 included in training module 108 occurs prior to utilizing system 100 to classify species of microbes 124 captured in mixture image(s) 118. Additionally, machine learning model 134 can continuously be trained by performing processes discussed herein (e.g., process 202), and/or utilizing mixture image(s) 118, mixture sample 120, and classifications rendered by classification module 138, as discussed herein (e.g., process 200). In exemplary embodiments, machine learning model 134 is using a plurality of training images 140. Training images 140 include images (see, FIG. 4), similar to mixture image(s) 118, of training mixtures 142 (see, FIG. 4). Each training mixture 142 used to generate training images 140 includes and/or is formed from a plurality of training fluorophore-DNA probes 144 and a plurality of training species of microbes 146. Each of the plurality of training fluorophore-DNA probes 144 correspond to and/or are associated with a single training species of microbe of the plurality of training species of microbes 146. That is, each of the plurality of training fluorophore-DNA probes 144 includes a DNA code sequence that corresponds to, is associated with, and/or complimentary to a DNA code sequence (e.g., 16S rRNA gene sequence) of a known training species of microbe included in the plurality of training species of microbes 146. As such, the number of training species of microbes 146 used to form training mixtures 142 for training machine learning model 134 is equal to the number of training fluorophore-DNA probes 144, and therefore when mixed together, a single training fluorophore-DNA probe 144 will ideally bond with a single microbe forming the training species of microbes 146.

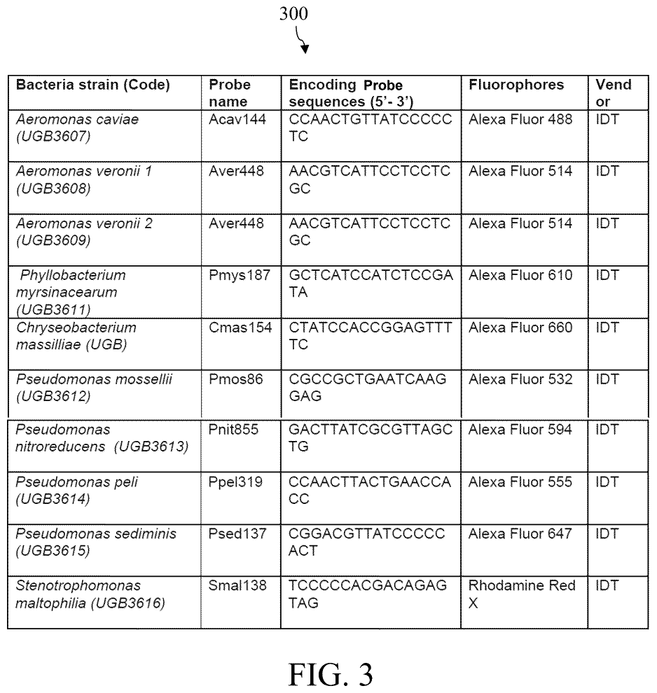

As discussed herein, training images 140 used to train machine learning model 134 include an image of training mixture 142 including each of the plurality of training fluorophore-DNA probes 144 and a single species of microbe forming the training species of microbes 146. That is, the training images 140 received by system 100 for training purposes includes a plurality of images where each image includes a distinct training mixture 142 formed from every training fluorophore-DNA probe 144 mixed with a single or isolated species of microbe included in the training species of microbes 146. In exemplary embodiments where ten (10) training species of microbes 146 and ten (10) corresponding training fluorophore-DNA probes 144 are utilized to train machine learning model 134, ten (10) distinct training images 140 are received by system 100 for training purposes (see, FIGS. 3 and 4).

In exemplary embodiments, analyzer model 148 interacts and/or communicates with various components and/or models within training module 108 to analyze training images 140 and identify bonds between training fluorophore-DNA probes 144 and the isolated species of microbe included in each training mixture 142. For example, analyzer model 148 is configured to analyze the received training images 140 to identify bonds between at least one of the training fluorophore-DNA probes 144 and the isolated species of microbe of the plurality of training species of microbes 146 used to form training mixtures 142. In the exemplary embodiment, and as discussed herein, it is predetermined that at least one of the plurality of training fluorophore-DNA probes 144, specifically the training fluorophore-DNA probe 144 corresponding to isolated species of microbe, included in training mixture 142 will bond with the isolated species of microbe also included in the training mixture 142. However, and as discussed herein, more than one training fluorophore-DNA probe 144 may bond with the isolated species of microbe included in the training mixture 142 due to, for example, close, but not identical, DNA code sequences/16S rRNA gene sequences for multiple training fluorophore-DNA probe 144/training species of microbes 146.

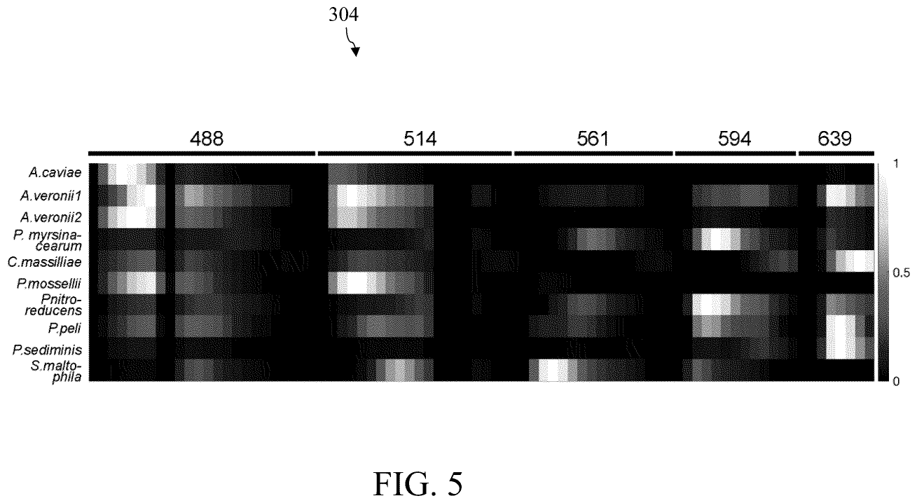

Additionally, and in non-limiting examples, training images 140 analyzed by analyzer model 148, as discussed herein, can include a plurality of images or profiles imaged under various operational parameters. For example, and as discussed herein (see e.g., FIG. 5), in addition to capturing images of training mixtures 142, a spectra profile for each training mixture 142 is generated and analyzed to aid in training machine learning model 134. More specifically, a spectra profile for each training mixture 142 is generated, created, and/or established by exciting the training fluorophore-DNA probes 144, and more particularly the fluorophore-based dye included in the training fluorophore-DNA probes 144, included in the training mixtures 142 using a range of wavelengths. In non-limiting examples, the fluorophore-based dye included in the training fluorophore-DNA probes 144 mixed with an isolated training species of microbe is exposed to and/or excited using a laser or multiple lasers producing various wavelengths between approximately 400 nanometers (nm) and approximately 700 nm. The generated spectra profile for each training mixture 142 allows for the production of a heatmap (see, FIG. 5) that represents detected or identified bonds between at least one of the plurality of training fluorophore-DNA probes 144 and each isolated training species of microbe included in the training mixture 142.

Cross-hybridization bond model 150 (hereafter, “CHB model 150”) interacts and/or communicates with various components and/or models within training module 108 to identify a pattern of cross-hybridization bonds within training mixtures 142. That is, CHB model 150, utilizing training images 140 and/or bonds identified by analyzer model 148, identifies, defines, and/or establishes a pattern of cross-hybridization bonds between at least one training fluorophore-DNA probe 144 and the isolated species of microbe of the plurality of training species of microbes 146 included in the training mixtures 142. In exemplary embodiments, the pattern of cross-hybridization bonds identified by CHB model 150 includes bonds between at least one training fluorophore-DNA probe 144 in training mixtures 142 that does not correspond to the one or isolated training species of microbe of the plurality of training species of microbes 146 also included in the training mixture 142. Additionally, or alternatively, CHB model 150 utilizes the generated spectra profile to aid in the identification of the pattern of cross-hybridization bonds occurring with training mixtures 142. For example, the generated heatmap depicting a light intensity at each excitation-wavelength can aid in identifying the various bonds, including cross-hybridization bonds, formed between training fluorophore-DNA probe 144 and the isolated species of microbe of the plurality of training species of microbes 146 included in the training mixtures 142.

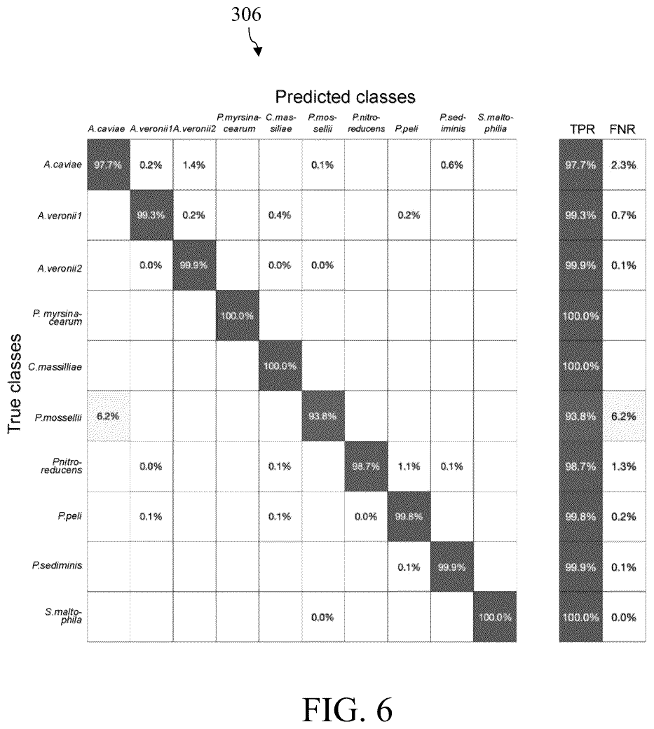

Validation model 152 interacts and/or communicates with various components and/or models within training module 108 to validate that bonds are formed between predetermined training fluorophore-DNA probe 144 and each of the plurality of training species of microbes 146 included in the training mixtures 142. Utilizing predetermined characteristics relating to the training fluorophore-DNA probes 144 and each of the plurality of training species of microbes 146 included in the training mixtures 142, training images 140, generated spectra profiles for training mixtures 142, and the identified pattern of cross-hybridization bonds within training mixtures 142, validation model 152 classifies each training species of microbes 146 and confirms that each training fluorophore-DNA probe 144 is bonded with the corresponding and/or complementary training species of microbes 146 within analyzed training mixtures 142. In an exemplary embodiment, and utilizing the known and/or identified information (e.g., pattern of cross-hybridization bonds, generated spectra profile), a confusion matrix (see, FIG. 6) is generated to validate the accuracy of predicting the classification of each training species of microbes 146 included in training mixtures 142. Although a confusion matrix is discussed herein for validating the accuracy of predicting the classification of each training species of microbes 146 included in training mixtures 142, it is understood that any suitable mathematical validation or classification concept can be used. Furthermore, the training module 108, and specifically validation model 152, utilizes any suitable machine learning technique and/or processing for validating and/or classifying each of the respective training species of microbes 146. For example, validation model 152 utilizes a support vector machine learning technique and/or a support vector machine classification model to validate and/or classify each of the training species of microbes 146 within training images 140. Validation model 152 is also configured to provide the confusion matrix or other validation output to machine learning model 134, along with the known and/or identified information (e.g., identified bond, identified pattern of cross-hybridization bonds, generated spectra profile), for training purposes.

Machine learning model 134 is trained based on the (i) known information about training mixtures 142 and its composition, (ii) training images 140, (iii) identified bonds between training fluorophore-DNA probes 144 and the plurality of training species of microbes 146 included in the training mixtures 142, (iv) the generated spectra profile for each training mixture 142, (v) the identified pattern of cross-hybridization bonds within each training mixture 142, and/or (vi) the validation of accuracy for classifying each training species of microbes 146 (e.g., confusion matrix).

FIG. 2A shows a flow chart illustrating processes 200 for classifying microbes. More specifically, processes 200 include classifying various microbes in a single sample using deoxyribonucleic acid (DNA) probes, synthesized fluorophore-based dyes, and machine learning models.

In process 202, a machine learning model is trained to aid in the classification of species of microbes. More specifically, a machine learning model is trained using a plurality of training fluorophore-DNA probes and a plurality of training species of microbes. As discussed herein with respect to FIG. 2B, each of the plurality of training fluorophore-DNA probes corresponds to a single training species of microbe of the plurality of training species of microbes. Additionally, and as discussed herein, the machine learning model is utilized in performing and/or aiding in the performance of additional processes (e.g., processes 206, 210) for classifying various species of microbes.

In process 204, at least one image of a mixture is received. More specifically, at least one image of a mixture sample of a plurality of fluorophore deoxyribonucleic acid (DNA) probes and an organic sample including a plurality of species of microbes are received. Each of the plurality of fluorophore-DNA probes corresponds to a predetermined species of microbe-which may or may not be included in the plurality of species of microbes included in the organic sample. In exemplary embodiments, each of the plurality of fluorophore DNA probes included in the mixture includes a distinct DNA code sequence complementary to and configured to bond with the predetermined species of microbe, as well as a uniquely colored, fluorophore-based dye.

In process 206, the received image of the mixture or mixture sample is analyzed. More specifically, the received image is analyzed to identify a bond between at least one fluorophore-DNA probe of the plurality of fluorophore-DNA probes and at least one species of microbe of the plurality of species of microbes. In an exemplary embodiment where one fluorophore-DNA probe of the plurality of fluorophore-DNA probes bonds to more than one species of microbe, the analyzing of the image also includes a first bond between a first fluorophore-DNA probe of the plurality of fluorophore-DNA probes and a first species of microbes of the plurality of species of microbes. Additionally in the exemplary embodiment, the analyzing of the image can include identifying a second bond between the first fluorophore-DNA probe of the plurality of fluorophore-DNA probes and a second species of microbes of the plurality of species of microbes. In the example, the second species of microbes is distinct from the first species of microbes.

In process 208, shown in phantom as optional, an accuracy percentage associated with the identified bond or bonds between fluorophore-DNA probe(s) and species of microbes of the plurality of species of microbes is determined. That is, an accuracy percentage for each bond between one of the plurality of fluorophore-DNA probe(s) and one species of microbes of the plurality of species of microbes is determined, calculated, and/or generated, where the accuracy percentage represents the likelihood that the species of microbes bonded to the fluorophore-DNA probe is the same as the predetermined species of microbe associated with, and/or complementary to the fluorophore-DNA probe. The accuracy percentage is determined using any suitable mathematical-validation or classification concept (e.g., confusion matrix), and/or any machine learning technique (e.g., support vector machine learning technique). Continuing the example discussed in process 206, determining the accuracy percentage includes determining a first accuracy percentage associated with the identified first bond between the first fluorophore-DNA probe of the plurality of fluorophore-DNA probes and the first species of microbes of the plurality of species of microbes. Additionally, and continuing the example, determining the accuracy percentage in process 208 also includes determining a second accuracy percentage associated with the identified second bond between the first fluorophore-DNA probe of the plurality of fluorophore-DNA probes and the second species of microbes of the plurality of species of microbes.

In process 210, the at least one species of microbe bonded with one or more fluorophore-DNA probes is classified. More specifically, the at least one species of microbe is classified, labelled, and/or identified based upon the predetermined species of microbe associated with the at least one fluorophore-DNA probe bonded with the at least one species of microbe. That is, where a species of microbe is bonded with a fluorophore-DNA probe, the bonded species of microbe is classified and/or identified as being the same as the predetermined species of microbe associated with the fluorophore-DNA probe to which the microbe is bonded. Additionally, or alternatively, the accuracy percentage determined in process 208 (shown in phantom as optional) aids in classifying the bonded species of microbe. For example, where the determined accuracy percentage is equal to or above a threshold (e.g., 90%), the bonded species of microbe is classified and/or identified as the predetermined species of microbe associated with the fluorophore-DNA probe the microbe is bonded to. Moreover, and continuing the example where a single fluorophore-DNA probe is bonded to multiple species of microbes, classifying at least one species of microbes includes identifying the first species of microbes as the predetermined species of microbe associated with the first fluorophore-DNA probe in response to the first accuracy percentage associated with the identified first bond being greater than the second accuracy percentage associated with the second bond.

FIG. 2B shows a flow chart illustrating process 202 for training an artificial intelligence model and/or a machine learning model. More specifically, process 202 includes training an artificial intelligence model and/or a machine learning model using training images of training mixtures to aid in processes (e.g., process 200) for classifying species of microbes.

In process 212, a plurality of training species of microbes are defined. That is, a predetermined number of species of microbes are defined, grouped, and/or identified as the plurality of training species of microbes that are used to train an artificial intelligence model and/or a machine learning model utilized to classify microbes in an organic sample. Defining the plurality of training species of microbes also includes defining a DNA code sequence (e.g., 16S rRNA gene sequence) of the plurality of training species of microbes.

In process 214, a plurality of training fluorophore-DNA probes are created. More specifically, a plurality of training fluorophore-DNA probes that are complementary to and/or correspond with the plurality of training species of microbes are created, synthesized, and/or manufactured. Each of the plurality of training fluorophore-DNA probes corresponds to and/or is associated with a single training species of microbe of the plurality of training species of microbes. That is, each of the plurality of training fluorophore-DNA probes includes a DNA code sequence that corresponds to, is associated with, and/or is complementary to a DNA code sequence (e.g., 16S rRNA gene sequence) of a known training species of microbe included in the plurality of training species of microbes. The DNA code sequence of each training fluorophore-DNA probe is complementary to the DNA code sequence of one of the training species of microbes to facilitate the bonding between the two when forming a training mixture (see, process 216). As such, the number of training species of microbes is equal to the number of training fluorophore-DNA probes. In addition to including a complementary DNA code sequence, each training fluorophore-DNA probe also includes a uniquely colored, fluorophore-based dye chemically conjugated with the DNA code sequence.

In process 216, training mixtures are formed. More specifically, a plurality of training mixtures are formed, created, and/or generated from the fluorophore-DNA probes and training species of microbes. In exemplary embodiments, each of the training mixtures is formed from one training species of microbe of the plurality of training species of microbes and the plurality of training fluorophore-DNA probes. As such, forming the training mixtures includes forming a plurality of training mixtures, where the number of training mixtures formed is equal to the number of species of microbes in the plurality.

In process 218, the plurality of training images of the training mixtures are received. More specifically, the plurality of training images are received, where each of the training images includes a training image of a mixture of the plurality of training fluorophore-DNA probes and one training species of microbes of the plurality of training species of microbes. Additionally, in exemplary embodiments, each of the plurality of training fluorophore-DNA probes included in each training mixture includes the distinct DNA code sequence complementary to and configured to bond with one of the training species of microbes, as well as the uniquely colored, fluorophore-based dye.

In process 220, each of the plurality of training images is analyzed. More specifically, each of the received training images is analyzed to identify at least one bond between at least one fluorophore-DNA training probe of the plurality of training fluorophore-DNA probes and the one training species of microbes of the plurality of training species of microbes. In an exemplary embodiment, at least one fluorophore-DNA training probe of the plurality of fluorophore-DNA training probes bonds to the one or isolated training species of microbe of the plurality of training species of microbes in each training mixture.

In process 222, a pattern of cross-hybridization bond(s) is identified. More specifically, a pattern of cross-hybridization bonds based on the identified at least one bond between at least one fluorophore-DNA training probe of the plurality of training fluorophore-DNA probes and the one training species of microbes of the plurality of training species of microbes is identified, determined, and/or established. In exemplary embodiments, the pattern of cross-hybridization bonds includes bonds between at least one training fluorophore-DNA probe that does not correspond to the one training species of microbes of the plurality of training species of microbes included in the training mixture.

In process 224, a machine learning model is trained using the identified bond and the identified cross-hybridization bonds. More specifically, and based on the analysis in process 220, and the identifying in process 222, a machine learning model is trained based on all identified bonds and all identified cross-hybridization bonds. In an exemplary embodiment, the machine learning model is trained with the identified bonds and the identified cross-hybridization bonds using a suitable mathematical validation or classification technique (e.g., confusion matrix), and/or any suitable machine learning technique and/or processing (e.g., a support vector machine learning technique and/or a support vector machine classification model).

FIGS. 3-7 depict various reference embodiments used to train a machine learning model (e.g., machine learning model 134; FIG. 1) to aid in the classification of various species of microbes. More specifically, the various reference embodiments shown in FIGS. 3-7 are used by training module 108 of system 100 shown in FIG. 1 to perform process 202 discussed herein with respect to FIGS. 2A and 2B to train machine learning model(s). As discussed herein, training machine learning model(s) using processes 202 improves the accuracy for system 100 when classifying species of microbes included in an imaged, organic sample (e.g., mixture sample 120). It is understood that similarly numbered and/or named components may function in a substantially similar fashion. Redundant explanation of these components has been omitted for brevity.

FIG. 3 shows a table 300 including information relating to a plurality of training species of microbes 146 and training fluorophore-DNA probes 144 used to train machine learning model 134 (see, FIG. 1). More specifically, table 300 shown in FIG. 3 includes a predetermined group of training species of microbes 146 (e.g., Column 1: Bacteria strain), as well as the identified, created, and/or manufactured plurality of training fluorophore-DNA probes 144 (e.g., Columns 2-4). Each probe of the plurality of training fluorophore-DNA probes 144 corresponds and/or complements an individual species of microbe forming the plurality of training species of microbes 146. As shown in FIG. 3, each of the plurality of training fluorophore-DNA probes 144 includes and/or is formed from a DNA code sequence (e.g., Column 3: Encoding Probe Sequence) that is configured to bond with a DNA sequence of the corresponding training species of microbe 146, as well as a uniquely colored, fluorophore-based dye (e.g., Column 4: Fluorophores). As discussed herein, each training species of microbes 146 are predetermined and/or preselected for the purposes of training machine learning model 134, while each of the plurality of training fluorophore-DNA probes 144 are manufactured and/or created based on the training species of microbes 146.

FIG. 4 shows a plurality of images 302 captured of various mixtures of the training microbes and the training fluorophore-DNA probes included in the table of FIG. 3. For example, FIG. 4 depicts various non-limiting examples of training images 140 (see, FIG. 1) of training mixtures 142 including the plurality of training fluorophore-DNA probes 144 and training species of microbes 146. As shown, the y-axis represents the plurality of training fluorophore-DNA probes 144, while the x-axis represents each microbe included in the plurality of training species of microbes 146. In the example shown in FIG. 4, and as similarly discussed herein, images 302/training images 140 are generated and/or captured using training mixtures 142. Specifically, each of the plurality of training fluorophore-DNA probes 144 (e.g., y-axis) are mixed with each isolated training species of microbe 146 (e.g., x-axis), and images 302/training images 140 are generated. The illuminated portions of each image 302/training image 140 represent a bond between the isolated, training species of microbe 146 and each training fluorophore-DNA probe 144.

FIG. 5 shows spectra profiles 304 for various training mixtures 142 of training species of microbes 146 and the training fluorophore-DNA probes 144. Spectra profile 304 includes a heatmap that represents detected or identified bonds between at least one of the plurality of training fluorophore-DNA probes 144 and each isolated training species of microbe 146 included in the training mixture 142. That is, the light intensity of each pixel included in the spectra profile 304 represents the strength and/or number of bonds formed between the plurality of training fluorophore-DNA probes 144 and each of the training species of microbes 146, as represented in the y-axis. As discussed herein, in order to generate spectra profile 304 each training mixture 142 is exposed to and/or excited using a laser or multiple lasers producing various wavelengths. The exposure to the various wavelengths is to excite the fluorophore-based dye included in the training fluorophore-DNA probes 144 and mixed with an isolated training species of microbe 146. Exciting the fluorophore-based dye in turn creates the heatmap depicted in spectra profile 304. The wavelengths of the laser used to excite the fluorophore-based dye included in the plurality of training fluorophore-DNA probes 144 are between approximately 400 nanometers (nm) and approximately 700 nm. For example, and as shown in FIG. 5, the wavelengths used to excite fluorophore-based dye include a plurality of predetermined or predefined wavelengths—488 nm, 514 nm, 561 nm, 594 nm, and 639 nm. The wavelengths may be dependent, at least in part, on the plurality of training fluorophore-DNA probes 144, and more specifically the fluorophore-based dyes included in the plurality of training fluorophore-DNA probes 144.

FIG. 6 shows a confusion matrix 306 depicting the accuracy of classifying the training species of microbes 146 using the training fluorophore-DNA probes 144. That is, and utilizing the known or predetermined information relating to the various portions forming training mixtures 142 as shown in table 300 (see FIG. 3), utilizing images 302/training images 140 (see, FIG. 4), and spectra profile 304 (see, FIG. 5), confusion matrix 306 depicts the accuracy of predicted classification for each training species of microbes 146. In exemplary embodiments, and as discussed herein, a support vector machine training technique and/or process can be performed to cross-validate the accuracies of classification as generated in the confusion matrix 306.

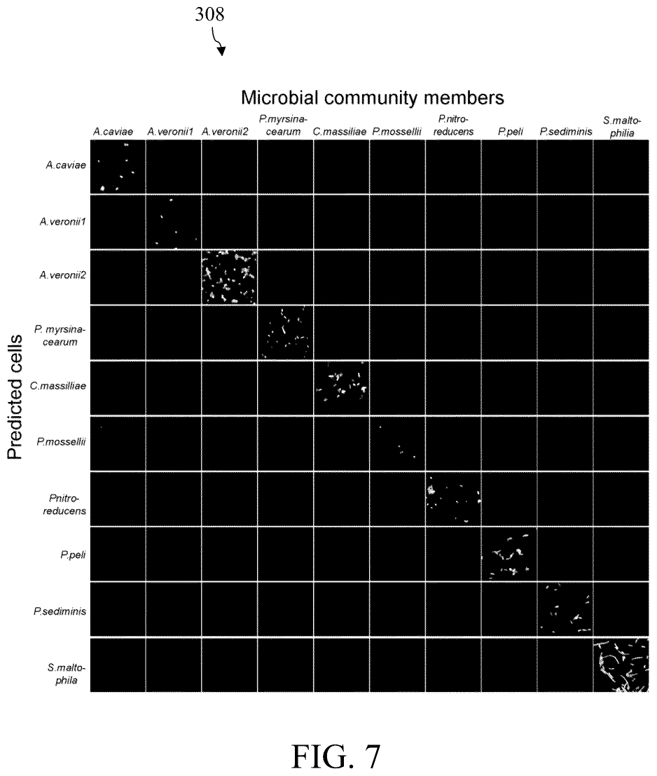

FIG. 7 shows various adjusted images 308 depicting each training species of microbes 146 and the corresponding training fluorophore-DNA probes 144. In the exemplary embodiment, adjusted images 308 shown in FIG. 7 are generated and/or modified after the generation of confusion matrix 306 and/or the determination of the accuracies of classification for each training species of microbes 146. Adjusted images 308, or a training classifier including the plurality of adjusted images 308, can be provided to machine learning model 134, along with the other embodiments (e.g., FIGS. 3-6) and utilized to train machine learning model 134, and/or aid in the classification of unknown species of microbes 124 included in mixture sample 120 (see, FIG. 1).

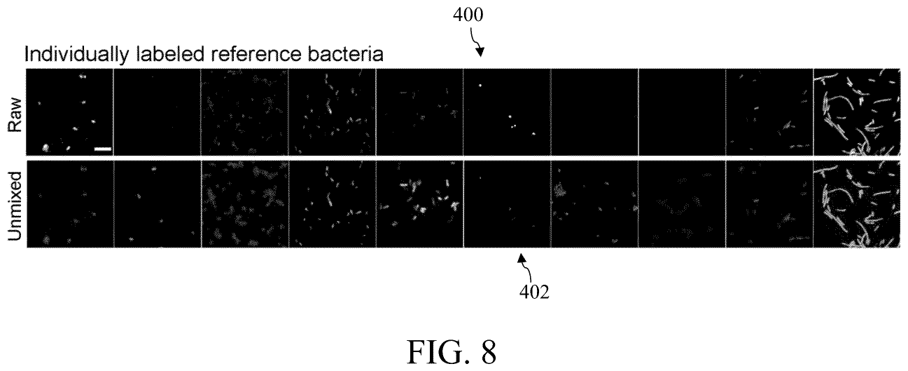

FIGS. 8 and 9 show various images of unclassified species of microbes 124 that utilize system 100 and/or processes 200 for classifying the species of microbes 124. More specifically, FIG. 8 shows various raw 400 and fluorophore-based dye highlighted images 402 of various, isolated species of microbes 124 prior to classification. Furthermore, FIG. 9 shows a fluorophore-based dye highlighted image 404 of a mixture (e.g., mixture sample 120) of the various species of microbes 124 included in FIG. 8, as well as the plurality of fluorophore-DNA probes 126 included in the mixture. As shown in FIG. 9, in following process 200, and utilizing fluorophore-DNA probes 126 including fluorophore-based dyes 130, the various species of microbes 124 included in the sample depicted in FIG. 9 are classified and/or uniquely colored for ease of detection.

FIG. 10 shows a table 500 emphasizing the improved accuracy in classifying species of microbes using processes 200 and/or system 100, as discussed herein. More specifically, table 500 shown in FIG. 10 includes various identified species of microbes (e.g., microbes 124), the accuracy in detecting or classifying the species of microbes using conventional linear techniques, and the accuracy in classifying species of microbes using the machine learning-based classification processes 200 of FIGS. 2A and 2B and/or system 100 shown in FIG. 1. By comparison, performing classification processes using deoxyribonucleic acid (DNA) probes, synthesized, fluorophore-based dyes, and machine learning, as similarly discussed herein with respect to FIG. 1-7, shows improvement for every single microbe listed in table 500 when compared to conventional linear techniques.

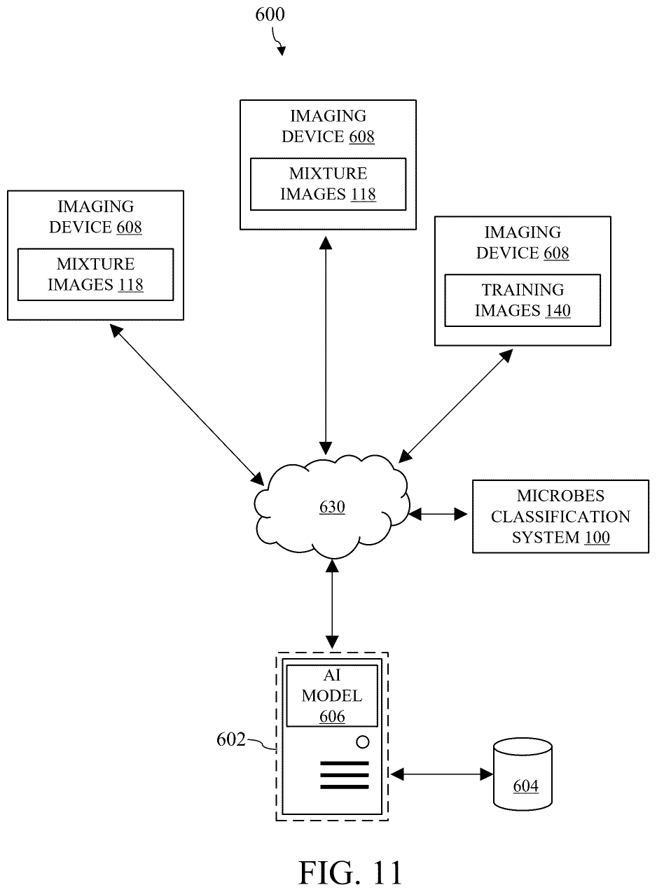

FIG. 11 is a schematic of a system 600 for training artificial intelligence models and/or machine learning models with improved accuracy information and/or data obtained from training images 140 (see, FIG. 1). As discussed herein, the system 600 may be included within and/or in communication with and utilized in conjunction with system 100 discussed herein with respect to FIG. 1. Additionally, and as discussed herein, system 600 may aid system 100 in classifying species of microbes 124 using mixture image(s) 118, as similarly discussed herein.

FIG. 11 illustrates components of a system 600 for training artificial intelligence models, in accordance with present implementations. The system 600 may include a remote server 602, a system database 604, artificial intelligence models 606, and imaging devices 608 (similar to image device 112 of FIG. 1) that capture and/or generate mixture images 118 and/or training images 140. For example, the system 600 may include one or more administrative computing devices that may be utilized to communicate with and configure various settings, parameters, or controls of the system 600. Various components depicted in FIG. 11 may be implemented to receive and process mixture images 118 and/or training images 140 to train the artificial intelligence models 606, which can subsequently be deployed to system 100 to assist in classifying species of microbes 124, as discussed herein. The above-mentioned components may be connected to each other through a network 630. Examples of the network 630 may include, but are not limited to, private or public local-area networks (LAN), wireless LAN (WLAN) networks, metropolitan area networks (MAN), wide-area networks (WAN), cellular communication networks, and the Internet. The network 630 may include wired and/or wireless communications according to one or more standards and/or via one or more transport mediums.

The system 600 is not confined to the components described herein and may include additional or other components, not shown for brevity, which are to be considered within the scope of the examples described herein.

The communication over the network 630 may be performed in accordance with various communication protocols such as Transmission Control Protocol and Internet Protocol (TCP/IP), User Datagram Protocol (UDP), and IEEE communication protocols. In one example, the network 630 may include wireless communications according to Bluetooth specification sets or another standard or proprietary wireless communication protocol. In another example, the network 630 may also include communications over a cellular network, including, e.g., a GSM (Global System for Mobile Communications), CDMA (Code Division Multiple Access), EDGE (Enhanced Data for Global Evolution) network.

Microbes classification system 100 and imaging devices 608 may be similar to, and include any of the structure and functionality of, the devices and/or systems shown and discussed herein with respect to FIG. 1. In exemplary embodiments, imaging device 608 transmits mixture image(s) 118 and/or training image(s) 140, which may be analyzed as discussed herein.

The remote server 602 may receive mixture image(s) 118 and/or training image(s) 140 during operation of the imaging device, and perform the processing techniques described herein to classify species of microbes and for training the artificial intelligence models 606 to aid the system in the classification process. For example, the remote server 602 can include, or implement any of the functionality of, machine learning model 134, analyzer model 148, CHB model 150, and/or validation model 152 of system 100 shown and discussed herein with respect to FIG. 1. For example, the remote server 602 may receive various mixture image(s) 118 and/or training image(s) 140 from various imaging devices 608, and store mixture image(s) 118 and/or training image(s) 140 in the system database 604.

The remote server 602 can implement the functionality described in connection with FIGS. 2A and 2B to classify species of microbes and to train machine learning model 134 to aid in the classification. The classification of the species of microbes using system 100 can be utilized as additional ground truth data for training the artificial intelligence model, which can be generated by the remote server 602 and stored in the system database 604.

The artificial intelligence models 606 may be stored in the system database 604 and may include artificial intelligence models that can classify the species of microbes in a sample mixture, as well as train a machine learning model 134 of the training module 108 within system 100. For example, the artificial intelligence models 606 can include the artificial intelligence model 136 of training module 108 for system 100 shown in FIG. 1. In some implementations, the artificial intelligence models 606 may be generated or trained for different types of mixture image(s) 118 and/or training image(s) 140 and/or classification processes. For example, the artificial intelligence models 606 may include multiple artificial intelligence models, each of which may be trained for a specific type of species of microbes (e.g., bacteria only). One or more of the artificial intelligence models 606 may be derived from a similar base model, which may be fine-tuned for particular applications.

The artificial intelligence models 606 can be or may include one or more neural networks. For example, the artificial intelligence models 606 can be a single shot multi-box detector, and can process mixture image(s) 118 and/or training image(s) 140 in one forward pass. Processing mixture image(s) 118 and/or training image(s) 140 in one forward pass improves processing efficiency, and enables the artificial intelligence models 606 to be utilized for real-time or near real-time classification processes. For example, the input to the artificial intelligence models 606 may be pre-processed, or the artificial intelligence models 606 itself may perform additional processing on the input data.

The remote server 602 can train one or more of the artificial intelligence models 606 using training data stored in the system database 604. In an example training process, the artificial intelligence models 606 can be trained using a set of training data that includes training image(s) 140, as well as previously generated mixture image(s) 118. Training image(s) 140 used in the training data may be generated by imaging device 608 to facilitate supervised learning. For example, at least a portion of the ground truth data can be generated by the remote server 602 using processes and/or techniques described herein. The training data may also be pre-processed via any suitable data augmentation approach (e.g., normalization, encoding, any combination thereof, etc.) to produce a dataset with modified properties to improve model generalization using the ground truth.

The remote server 602 can train an artificial intelligence model 606, for example, by performing supervised learning techniques to adjust the parameters of the artificial intelligence model 606 based on a loss computed from the output generated by the artificial intelligence model 606 and ground truth data corresponding to the input provided to the artificial intelligence model 606. Inputs to the artificial intelligence model 606 may include mixture image(s) 118 and/or training image(s) 140 obtained during operation of imaging devices 608, and stored in the system database 604. The artificial intelligence model 606 may be trained on a portion of the training data using a suitable optimization algorithm, such as stochastic gradient descent. The remote server 602 can train the artificial intelligence model 606 by minimizing the calculated loss function by iteratively updating the trainable parameters of the artificial intelligence model 606 (e.g., using backpropagation, etc.). The remote server 602 can evaluate the artificial intelligence model 606 on a held-out portion of the training data (e.g., validation set that was not used to train the artificial intelligence model 606) to assess the performance of the artificial intelligence model 606 on unseen data. The evaluation metrics used to assess the model's performance may include accuracy, precision, recall, and F1 score, among others. Furthermore, and as discussed herein, the artificial intelligence model 606 may be trained using support vector machine learning techniques.

The remote server 602 can train an artificial intelligence model 606 until a training termination condition is met. Some non-limiting training termination conditions include a maximum number of iterations being met or a predetermined performance threshold being met. The performance threshold can be satisfied when the artificial intelligence model 606 reaches a certain level of accuracy, F1 score, precision, recall, or any other relevant metric on a validation set. The remote server 602 can provide the trained artificial intelligence model 606 to system 100 for which the artificial intelligence model 606 was trained.

The remote server 602 can update one or more of the artificial intelligence models 606 (e.g., by retraining, fine-tuning, or other types of training processes) when a plurality of mixture image(s) 118 and/or training image(s) 140 are received and utilized to produce additional training data. The remote server 602 (or system 100; FIG. 1) can generate the additional training data by determining corrections to classifications made by the artificial intelligence model. Although the artificial intelligence models 606 can include neural networks trained using supervised learning techniques, it should be understood that any alternative and/or additional machine learning model(s) may be used to implement similar learning engines.

An example technical effect of the systems, methods, and program products described herein includes improved classification of different species of microbes using deoxyribonucleic acid (DNA) probes, synthesized, fluorophore-based dyes, and machine learning.

Some embodiments involve the use of one or more electronic processing or computing devices. As used herein, the terms “processor” and “computer” and related terms, e.g., “processing device,” and “computing device” are not limited to just those integrated circuits referred to in the art as a computer, but broadly refer to a processor, a processing device or system, a general purpose central processing unit (CPU), a graphics processing unit (GPU), a microcontroller, a microcomputer, a programmable logic controller (PLC), a reduced instruction set computer (RISC) processor, a field programmable gate array (FPGA), a digital signal processor (DSP), an application specific integrated circuit (ASIC), and other programmable circuits or processing devices capable of executing the functions described herein, and these terms are used interchangeably herein. These processing devices are generally “configured” to execute functions by programming or being programmed, or by the provisioning of instructions for execution. The above examples are not intended to limit in any way the definition or meaning of the terms “processor,” “processing device,” and related terms.

The various aspects illustrated by logical blocks, modules, circuits, processes, algorithms, and algorithm steps described above may be implemented as electronic hardware, software, or combinations of both. Certain disclosed components, blocks, modules, circuits, and steps are described in terms of their functionality, illustrating the interchangeability of their implementation in electronic hardware or software. The implementation of such functionality varies among different applications given varying system architectures and design constraints. Although such implementations may vary from application to application, they do not constitute a departure from the scope of this disclosure.

Aspects of embodiments implemented in software may be implemented in program code, application software, application programming interfaces (APIs), firmware, middleware, microcode, hardware description languages (HDLs), or any combination thereof. A code segment or machine-executable instruction may represent a procedure, a function, a subprogram, a routine, a subroutine, a module, a software package, a class, or any combination of instructions, data structures, or program statements. A code segment may be coupled to, or integrated with, another code segment or an electronic hardware by passing or receiving information, data, arguments, parameters, memory contents, or memory locations. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the disclosed functions may be embodied, or stored, as one or more instructions or code on or in memory. In the embodiments described herein, memory includes non-transitory computer-readable/machine-readable media, which may include, but is not limited to, media such as flash memory, a random access memory (RAM), read-only memory (ROM), erasable programmable read-only memory (EPROM), electrically erasable programmable read-only memory (EEPROM), and non-volatile RAM (NVRAM). As used herein, the term “non-transitory computer-readable media” is intended to be representative of any tangible, computer-readable media, including, without limitation, non-transitory computer storage devices, including, without limitation, volatile and non-volatile media, and removable and non-removable media such as firmware, physical and virtual storage, CD-ROM, DVD, and any other digital source such as a network, a server, cloud system, or the Internet, as well as yet to be developed digital means, with the sole exception being a transitory propagating signal. The methods described herein may be embodied as executable instructions, e.g., “software” and “firmware,” in a non-transitory computer-readable medium. As used herein, the terms “software” and “firmware” are interchangeable and include any computer program stored in memory for execution by personal computers, workstations, clients, and servers. Such instructions, when executed by a processor, configure the processor to perform at least a portion of the disclosed methods.

The foregoing drawings show some of the processing associated with several embodiments of this disclosure. In this regard, each drawing or block within a flow diagram of the drawings represents a process associated with embodiments of the method described. It should also be noted that in some alternative implementations, the acts noted in the drawings or blocks may occur out of the order noted in the figure or, for example, may in fact be executed substantially concurrently or in the reverse order, depending upon the act involved. Also, one of ordinary skill in the art will recognize that additional blocks that describe the processing may be added.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately” and “substantially,” are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise. “Approximately” and/or “substantially” as applied to a particular value of a range applies to both values, and unless otherwise dependent on the precision of the instrument measuring the value, may indicate +/−10% of the stated value(s).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed is:

1. A method, comprising:
receiving an image of a mixture of a plurality of fluorophore deoxyribonucleic acid (DNA) probes and an organic sample including a plurality of species of microbes,
wherein each of the plurality of fluorophore-DNA probes corresponds to a predetermined species of microbe;
analyzing, by one or more processors, the received image of the mixture to identify a bond between at least one fluorophore-DNA probe of the plurality of fluorophore-DNA probes and at least one species of microbe of the plurality of species of microbes; and
classifying, by the one or more processors, the at least one species of microbe based upon the predetermined species of microbe associated with the at least one fluorophore-DNA probe bonded with the at least one species of microbe.

2. The method of claim 1, wherein the analyzing of the received image of the mixture further includes at least one of:
identifying, by the one or more processors, a first bond between a first fluorophore-DNA probe of the plurality of fluorophore-DNA probes and a first species of microbes of the plurality of species of microbes, or
identifying, by the one or more processors, a second bond between the first fluorophore-DNA probe of the plurality of fluorophore-DNA probes and a second species of microbes of the plurality of species of microbes, the second species of microbes distinct from the first species of microbes.

3. The method of claim 2, further comprising:
determining, by the one or more processors, a first accuracy percentage associated with the identified first bond between the first fluorophore-DNA probe of the plurality of fluorophore-DNA probes and the first species of microbes of the plurality of species of microbes; and
determining, by the one or more processors, a second accuracy percentage associated with the identified second bond between the first fluorophore-DNA probe of the plurality of fluorophore-DNA probes and the second species of microbes of the plurality of species of microbes.

4. The method of claim 3, wherein the classifying of the at least one species of microbe further includes:
identifying, by the one or more processors, the first species of microbes as the predetermined species of microbe associated with the first fluorophore-DNA probe in response to the first accuracy percentage associated with the identified first bond being greater than the second accuracy percentage associated with the second bond.

5. The method of claim 1, further comprising:
training, by the one or more processors, a machine learning model using a plurality of training fluorophore-DNA probes and a plurality of training species of microbes, each of the plurality of training fluorophore-DNA probes corresponding to a single training species of microbes of the plurality of training species of microbes,
wherein the machine learning model is configured to:
analyze the received image of the mixture, and
classify the at least one species of microbe of the plurality of species of microbes.

6. The method of claim 5, wherein the training of the machine learning model further includes:
receiving a plurality of training images, each of the plurality of training images including a training image of a mixture of the plurality of training fluorophore-DNA probes and one training species of microbes of the plurality of training species of microbes;
analyzing, by the one or more processors, each of the plurality of received training images to identify at least one bond between at least one fluorophore-DNA training probe of the plurality of training fluorophore-DNA probes and the one training species of microbes of the plurality of training species of microbes;
identifying, by the one or more processors, a pattern of cross-hybridization bonds based on the identified at least one bond between at least one fluorophore-DNA training probe of the plurality of training fluorophore-DNA probes and the one training species of microbes of the plurality of training species of microbes,
wherein the pattern of cross-hybridization bonds includes bonds between at least one training fluorophore-DNA probe that does not correspond to one training species of microbes of the plurality of training species of microbes; and
training, by the one or more processors, the machine learning model using the identified at least one bond and the identified pattern of cross-hybridization bonds.

7. The method of claim 1, wherein each of the plurality of fluorophore DNA probes included in the mixture includes:
a distinct DNA code sequence complementary to and configured to bond with the predetermined species of microbe; and
a uniquely colored, fluorophore-based dye.

8. A system comprising:
one or more processors coupled to non-transitory memory, the one or more processors configured to:
receive an image of a mixture of a plurality of fluorophore deoxyribonucleic acid (DNA) probes and an organic sample including a plurality of species of microbes,
wherein each of the plurality of fluorophore-DNA probes corresponds to a predetermined species of microbe;
analyze the received image of the mixture to identify a bond between at least one fluorophore-DNA probe of the plurality of fluorophore-DNA probes and at least one species of microbe of the plurality of species of microbes; and
classify the at least one species of microbe based upon the predetermined species of microbe associated with the at least one fluorophore-DNA probe bonded with the at least one species of microbe.

9. The system of claim 8, wherein the one or more processors analyzing the received image of the mixture are further configured to at least one of:
identify a first bond between a first fluorophore-DNA probe of the plurality of fluorophore-DNA probes and a first species of microbes of the plurality of species of microbes; or
identify a second bond between the first fluorophore-DNA probe of the plurality of fluorophore-DNA probes and a second species of microbes of the plurality of species of microbes, the second species of microbes distinct from the first species of microbes.

10. The system of claim 9, wherein the one or more processors are further configured to:
determine a first accuracy percentage associated with the identified first bond between the first fluorophore-DNA probe of the plurality of fluorophore-DNA probes and the first species of microbes of the plurality of species of microbes; and
determine a second accuracy percentage associated with the identified second bond between the first fluorophore-DNA probe of the plurality of fluorophore-DNA probes and the second species of microbes of the plurality of species of microbes.

11. The system of claim 10, wherein the one or more processors classifying the at least one species of microbe are further configured to:
identify the first species of microbes as the predetermined species of microbe associated with the first fluorophore-DNA probe in response to the first accuracy percentage associated with the identified first bond being greater than the second accuracy percentage associated with the second bond.

12. The system of claim 8, wherein the one or more processors are further configured to:
train a machine learning model using a plurality of training fluorophore-DNA probes and a plurality of training species of microbes, each of the plurality of training fluorophore-DNA probes corresponding to a single training species of microbes of the plurality of training species of microbes,
wherein the machine learning model is configured to:
analyze the received image, and
classify the at least one species of microbe of the plurality of species of microbes.

13. The system of claim 12, wherein the one or more processors training the machine learning model are further configured to:
receive a plurality of training images, each of the plurality of training images including a training image of a mixture of the plurality of training fluorophore-DNA probes and one training species of microbes of the plurality of training species of microbes,
analyze each of the plurality of received training images to identify at least one bond between at least one fluorophore-DNA training probe of the plurality of training fluorophore-DNA probes and the one training species of microbes of the plurality of training species of microbes,
identify a pattern of cross-hybridization bonds based on the identified at least one bond between at least one fluorophore-DNA training probe of the plurality of training fluorophore-DNA probes and the one training species of microbes of the plurality of training species of microbes, and
train the machine learning model using the identified at least one bond and the identified pattern of cross-hybridization bonds.

14. The system of claim 8, wherein each of the plurality of fluorophore DNA probes included in the mixture includes a distinct DNA code sequence complementary to and configured to bond with the predetermined species of microbe, and a uniquely colored, fluorophore-based dye.

15. A non-transitory computer-readable medium including one or more instructions stored thereon and executable by a processor to:
receive an image of a mixture of a plurality of fluorophore deoxyribonucleic acid (DNA) probes and an organic sample including a plurality of species of microbes,
wherein each of the plurality of fluorophore-DNA probes corresponds to a predetermined species of microbe;
analyze the received image of the mixture to identify a bond between at least one fluorophore-DNA probe of the plurality of fluorophore-DNA probes and at least one species of microbe of the plurality of species of microbes; and
classify the at least one species of microbe based upon the predetermined species of microbe associated with the at least one fluorophore-DNA probe bonded with the at least one species of microbe.

16. The non-transitory computer-readable medium of claim 15, wherein the one or more instructions are further executable by the processor to at least one of:
identify a first bond between a first fluorophore-DNA probe of the plurality of fluorophore-DNA probes and a first species of microbes of the plurality of species of microbes; or
identify a second bond between the first fluorophore-DNA probe of the plurality of fluorophore-DNA probes and a second species of microbes of the plurality of species of microbes, the second species of microbes distinct from the first species of microbes.

17. The non-transitory computer-readable medium of claim 16, wherein the one or more instructions are further executable by the processor to:
determine a first accuracy percentage associated with the identified first bond between the first fluorophore-DNA probe of the plurality of fluorophore-DNA probes and the first species of microbes of the plurality of species of microbes; and
determine a second accuracy percentage associated with the identified second bond between the first fluorophore-DNA probe of the plurality of fluorophore-DNA probes and the second species of microbes of the plurality of species of microbes.

18. The non-transitory computer-readable medium of claim 17, wherein the one or more instructions are further executable by the processor to:
identify the first species of microbes as the predetermined species of microbe associated with the first fluorophore-DNA probe in response to the first accuracy percentage associated with the identified first bond being greater than the second accuracy percentage associated with the second bond.

19. The non-transitory computer-readable medium of claim 15, wherein the one or more instructions are further executable by the processor to:
train a machine learning model using a plurality of training fluorophore-DNA probes and a plurality of training species of microbes, each of the plurality of training fluorophore-DNA probes corresponding to a single training species of microbes of the plurality of training species of microbes,
wherein the machine learning model is configured to:
analyze the received image, and
classify the at least one species of microbe of the plurality of species of microbes.

20. The non-transitory computer-readable medium of claim 19, wherein the one or more instructions are further executable by the processor to:
receive a plurality of training images, each of the plurality of training images including a training image of a mixture of the plurality of training fluorophore-DNA probes and one training species of microbes of the plurality of training species of microbes;
analyze each of the plurality of received training images to identify at least one bond between at least one fluorophore-DNA training probe of the plurality of training fluorophore-DNA probes and the one training species of microbes of the plurality of training species of microbes;
identify a pattern of cross-hybridization bonds based on the identified at least one bond between at least one fluorophore-DNA training probe of the plurality of training fluorophore-DNA probes and the one training species of microbes of the plurality of training species of microbes; and
train the machine learning model using the identified at least one bond and the identified pattern of cross-hybridization bonds.
